(19) United States
(12) Patent Application Publication
(10) Pub. No.: US 2012/0156286 A1
(43) Pub. Date: Jun. 21, 2012

(54) COMPOSITIONS AND METHODS FOR INCREASED DELIVERY OF COENZYME Q10

(75) Inventor: William P. SPENCER, Escondido, CA (US)

(73) Assignee: IMAGENETIX, INC., San Diego, CA (US)

(21) Appl. No.: 13/192,067

(22) Filed: Jul. 27, 2011

Related U.S. Application Data
(60) Provisional application No. 61/367,969, filed on Jul. 27, 2010.

(51) Int. Cl. A61K 31/122; A61P 39/06; A61K 9/48

(52) U.S. Cl. 424/452; 424/94.1; 514/690

(57) ABSTRACT

Disclosed are compositions comprising Coenzyme Q10 and a cetylated fatty acid blend, pharmaceutical formulations comprising the same and methods of increasing the systemic concentration of Coenzyme Q10 in an individual, comprising administering the same to the individual.

### RELATED APPLICATIONS

The present application claims priority to the U.S. Provisional Application Ser. No. 61/367,969, filed on Jul. 27, 2010, the entire disclosure of which is incorporated herein by reference.

### FIELD OF THE INVENTION

The present invention is in the field of pharmaceutical formulations, and in particular formulations developed to increase the absorption and bioavailability of Coenzyme Q10.

### BACKGROUND OF THE DISCLOSURE

Coenzyme Q10 (also known as CoQ10 or ubiquinone) is a naturally occurring compound that shares a structural similarity to vitamin K. It has a fundamental role in energy metabolism as a cofactor in the mitochondrial electron transport chain, and consequently is essential for the production of ATP, the energy currency of all cells. However, CoQ10, in its reduced form as the hydroquinone (also called ubiquinol), acts as a potent lipophilic antioxidant. There is evidence that CoQ10 also functions in cell signaling and gene expression. The structures of CoQ10 in both the oxidized and reduced form are shown below:

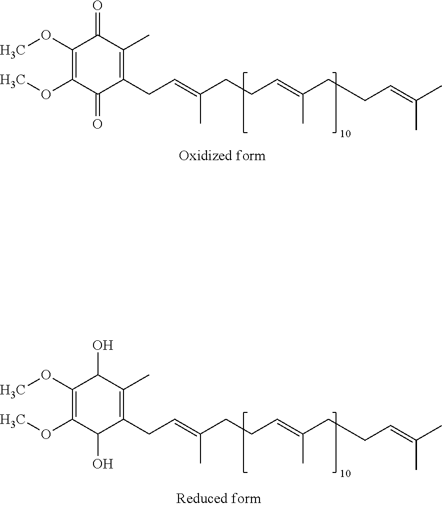

There is considerable interest in CoQ10 as a dietary supplement to improve health. CoQ10 is synthesized in the same pathway as cholesterol. It is believed that drugs that reduce cholesterol synthesis (i.e. statins) may also reduce endogenous CoQ10 synthesis, and thereby have a detrimental impact on a number of cellular processes. Further, given that CoQ10, in the reduced form, can act as an antioxidant, it may be that increasing circulating and tissue levels of CoQ10 by dietary supplementation may reduce oxidative stress.

One of the challenges to developing a dietary supplement of CoQ10 is that it is not well absorbed. Consequently, it would be desirable to develop a way to increase intestinal absorption of CoQ10 (increase bioavailability) in order to increase circulating and/or tissue concentrations of CoQ10.

### SUMMARY OF THE INVENTION

Disclosed are compositions comprising Coenzyme Q10 and a cetylated fatty acid. Also disclosed are pharmaceutical formulations comprising Coenzyme Q10 and a cetylated fatty acid. Further, disclosed are methods of increasing the systemic concentration of Coenzyme Q10 in an individual, comprising identifying an individual in need thereof and administering to the individual a composition comprising Coenzyme Q10 and a cetylated fatty acid.

### DETAILED DESCRIPTION OF THE EMBODIMENTS

The present inventors have previously disclosed a mixture of esterified fatty acids that has surprising therapeutic efficacy in treating certain inflammatory conditions. See, for example, U.S. Pat. No. 7,612,111, incorporated by reference herein in its entirety, and specifically the discussion therein on esterified fatty acids. As further discussed below, it is now discovered that the combination of one or more esterified fatty acids and Coenzyme Q10 (CoQ10) significantly increases the absorption of CoQ10 from the gastrointestinal track and thereby increase its systemic concentration.

Thus, in a first aspect, disclosed herein are compositions comprising Coenzyme Q10 and an esterified fatty acid. In some embodiments, the fatty acids are cetylated. In certain embodiments, the compositions comprise one esterified fatty acids, whereas in other embodiments, the compositions comprise a mixture of two or more cetylated fatty acids.

In some embodiments, the compositions comprise both esterified and non-esterified fatty acids.

In certain embodiments, the fatty acid is selected from the group consisting of decanoic acid, lauric acid, myristic acid, myristoleic acid, palmitoleic acid, palmitic acid, oleic acid, and stearic acid. In some of these embodiments, the cetylated fatty acid is selected from the group consisting of cetyl decanoate, cetyl laurate, cetyl myristate, cetyl myristoleate, cetyl palmitoleate, cetyl palmitate, cetyl oleate, and cetyl stearate.

In some embodiments, the mixture of esterified fatty acids is a composition similar to that disclosed in U.S. Pat. No. 7,612,111, which composition is sold under the trade name CELADRIN® (Imagenetix, San Diego, Calif.).

In other embodiments, the disclosed compositions comprises CellSorb® and CoQ10. CellSorb® is a mixture of esterified fatty acids that comprises one or more of the following esterified fatty acids: Myristic Acid, Myristoleic Acid, Palmitic Acid, Cetyl decanoate, Cetyl laurate, Cetyl myristate, Cetyl Myristoleate, Cetyl Palmitate, Cetyl palmitoleate, Cetyl stearate, Cetyl oleate, and Cetyl linoleate. In some embodiments, the CellSorb® composition comprises the following amounts of esterified fatty acids:

| (Esterified) Fatty Acid | Percentage by weight |
|---|---|
| Myristoleic Acid | 10-25% |
| Palmitic Acid | 1-10% |
| Cetyl decanoate | 1-5% |
| Cetyl laurate | 1-5% |
| Cetyl myristate | 20-40% |
| Cetyl Myristoleate | 10-25% |
| Cetyl Palmitate | 1-10% |
| Cetyl palmitoleate | 1-10% |
| Cetyl stearate | 1-10% |
| Cetyl oleate | 20-30% |
| Cetyl linoleate | 1-5% |

In certain embodiments, the composition further comprises a carrier or an excipient. Suitable carriers and excipients are well-known in the art. For example, in some embodiments, the carrier or excipient is selected from the group consisting of The lipid excipient can be selected from the group consisting of glyceryl behenate, glycerol esters of fatty acids, glyceryl dibehenate, behenoyl macrogoglycerides, glyceryl distearate, glycerol distearate, glyceryl palmitostearate, lauroyl macrogoglycerides, stearoyl macrogoglycerides, abitec products, glyceryl mono-oleate, medium chain mono-& diglycerides, glyceryl monocaprylate, glyceryl tricaprylate/caprate/stearate, hydrogenated vegetable oil, hydrogenated cottonseed oil, hydrogenated soybean oil, hydrogenated soybean oil and castor wax, polyoxyethylene 8 caprylic/capric glycerides, polyoxyethylene 6 caprylic/capric glycerides, polyoxyethylene 32 lauric glycerides, polyoxyethylene 6 prop. Glycol esters, polyoxyethylene 7 coconut glycerides, polyoxyethylene 30 coconut glycerides, polyoxyethylene 80 coconut glycerides, polyoxypropylene 15 stearyl ether, polyoxyethylene 26 glyceryl ether, polyoxyethylene 35 soybean glycerides, polyoxyethylene 20 sorbitol, polyoxypropylene 3 myristyl ether, polyoxypropylene 10 cetostearyl ether, palm kernelamide diethanolamide, triglycerol mono-oleate, sasol products, hydrogenated coco-glycerides, cetyl palmitate, trimyristin, tripalmitin, tristearin, hydrogenated palm oil, glyceryl monostearate, glyceryl stearate, cetearyl alcohol, cetyl alcohol, capric triglyceride, acetylated glycerides, glyceryl cocoate, and polyethylene glycol. In other embodiments, the carrier or excipient is selected from the group consisting of lecithin, olive oil, and tocopherol.

Generally, the amount of CoQ10 available in the compositions disclosed herein is sufficient to confer therapeutic benefit to the individual to whom the compositions is administered. In some embodiments, the composition comprises between 10-500 mg of Coenzyme Q10. In other embodiments, the composition comprises between 50-400 mg of Coenzyme Q10. In some specific embodiments, the composition comprises, 50, 100, 150, 200, 250 or 300 mg of Coenzyme Q10.

In some embodiments, the CoQ10 in the composition is predominantly (i.e., greater than about 75%, or greater than 85%, or greater than 90%, or greater than 95%) the reduced form of CoQ10, i.e., the hydroquinone form, also called ubiquinol. This form is shown to be therapeutically more advantageous than the oxidized form, i.e., the quinine form. In other embodiments, the CoQ10 in the composition comprises a mixture of the oxidized and the reduced forms.

In another aspect, disclosed herein are pharmaceutical formulations comprising Coenzyme Q10 and a cetylated fatty acid. By “pharmaceutical formulation” it is meant compositions disclosed above disposed in a formulation administrable to an individual. Formulations can take the form of push-fit capsules, soft capsules, dose sipping straws, sachets, or tablets.

In some embodiments, the formulation is in the form of a soft, sealed capsule. In some of these embodiments, the capsule is made of gelatin and a plasticizer. Examples of plasticizers include, but are not limited to, glycerol or sorbitol.

In another aspect, disclosed herein are methods of increasing the systemic concentration of Coenzyme Q10 in an individual, comprising identifying an individual in need thereof and administering to the individual a composition comprising Coenzyme Q10 and a cetylated fatty acid, wherein the composition is as disclosed above.

In some embodiments, the composition is administered once a day. In other embodiments, the composition is administered two or three times a day. In some embodiments, the unit dose of CoQ10 is about 100 mg. However, the administered dose is about 200 mg. Thus, in some embodiments, the administered dose of CoQ10 comprises two or more unit doses.

### EXAMPLES

The following examples are illustrative of some of the embodiments of the inventions disclosed herein and are not meant to be limiting.

### Example 1

### A Randomized, Crossover, Bioequivalence Study of 100 mg CoQ10/CellSorb Soft Gels Comparing with 3 Reference CoQ10 Products

Coenzyme Q10 (CoQ10) is a lipophilic molecule, which means that it is easily dissolved in oils, but not in water. This physical property of CoQ10 is part of the reason for its poor intestinal absorption in humans and animals, which is reported to be less than 10%. Because it is a lipophilic, vitamin-like substance, the use of CellSorb's unique CoQ10 delivery facilitates its entry into and utilization by the body. This study was performed to evaluate the performance of this new product.

### Study Design

This was an open label, randomized, crossover bioequivalence study of CellSorb-Q10 (CoQ10 mixed with CellSorb in comparison with three other commercially available CoQ10 products. Eleven, healthy adult males, meeting the necessary inclusion criteria and having no exclusions, were recruited for each of 3 groups (Total n=33). All groups and individuals were randomized as to the treatment and order of administration. After a 10 hr fast, two capsules (200 mg of CoQ10) were given either of one of the reference products or of CellSorb-Q10 (study designation=T). Subsequent doses of 2 capsules were given at the beginning of days 2 and 3. Participants were monitored for 96 hrs, with blood samples taken (14 during the first 24 hrs and samplings again at 48 hr, 72 hr and 96 hr). During this period, food and liquids were provided (minimal CoQ10 levels). At end of 96 hrs, participants were given a 7 day washout and then returned for the 2nd crossover treatment round. The three best selling CoQ10 products in the US market were chosen as reference and were designated as R1, R2 and R3 Ubiquinone. CoQ10 concentrations were analyzed using LC-MS/MS method. Blood chemistries were analyzed at the start of each treatment period. Thirty subjects completed the study, but 2 were eliminated due to compliance issues.

### Pharmacokinetic Terminology

AUC (area under the curve). When a substance is administered, its presence in the plasma can be analyzed at multiple time points and plotted. The AUC is the estimated sum of the areas under the plotted data points. The AUC timeframe analyzed is usually given as a subscript, such as AUC₀₋₂₄ (the first 24 hrs after administration).

Bioavailability. This is the rate and extent that a substance becomes available in the body's circulatory system.

Bioequivalence. Two treatments are said to be bioequivalent if there is no significant difference in bioavailability.

Cₘₐₓ. This is the maximum concentration of the substance observed in the plasma after administration of a dose. The peak or Cₘₐₓ for all treatments in this study occurs about 6 minutes after the administration of the dose. As indicated by relative degrees of absorption in the study results, Cₘₐₓ is related to the uptake of a substance after administration.

Tₘₐₓ. This is the time at which the Cₘₐₓ is observed. This provides an estimate of how quickly absorption is occurring and the substance getting into the circulation.

Crossover Study. This is a type of study in which a subject is first treated with one substance (test or reference) and then later treated with the other substance. This provides intra-individual comparisons.

### Study Results

As shown in the pharmacokinetic tables 4-6, CellSorb-Q10 demonstrated comparatively favorable results. The following clinical pharmacokinetic data and observations of the study are depicted below:

CellSorb-Q10 performed well pharmacokinetically in terms of bioavailability, bioequivalence and absorption, relative to the comparator products. The AUC, Tₘₐₓ and Cₘₐₓ results showed good delivery of CoQ10 into the system. Concentrations were high and at effective levels compared to the three reference products.

CellSorb-Q10 was bioequivalent to R1 and R2 Ubiquinone, and had superior bioavailability compared to R3 Ubiquinone. There was no significant difference between the AUC₀₋₂₄ₕᵣ and Cₘₐₓ average results for CellSorb-Q10 and the R1 and R2 Ubiquinones (p>0.3).

CellSorb-Q10 delivered greater AUC₀₋₂₄ₕᵣ and Cₘₐₓ response averages than R2 Ubiquinone. CellSorb-Q10 Mean AUC₀₋₂₄ₕᵣ was greater than either R2 Ubiquinone (by 10%) or R3 Ubiquinone (by 3.39 fold). And the mean Cₘₐₓ response for CellSorb-Q10 was 15% greater than that of R2 Ubiquinone.

CellSorb-Q10 delivered significantly greater bioavailability and absorption than the R3 Ubiquinone reference product. CellSorb-Q10 was bioequivalent to R1 and R2 in terms of Relative Degree of Absorption and Relative Bioavailability (Table 1). CellSorb-Q10 and reference products R1 and R2 showed supra-bioavailability compared with the R3 Ubiquinone.

### Relative Pharmacokinetic Relationships and Responses

This section shows the relative relationships between CellSorb-Q10, Ubiquinone R1, Ubiquinone R2 and Ubiquinone R3.

Relative Degree of Absorption (RDA): Reflects the uptake of CoQ10 from the dosing administered. This RDA compares Cₘₐₓ of CellSorb-Q10 compared to the values of the reference products. The results are shown in Table 1.

CellSorb-Q10 and R1 Ubiquinone were equivalent in the degree of CoQ10 absorption.

CellSorb-Q10 mean RDA was greater than R2 Ubiquinone & R3 Ubiquinone by 1.2× and 2.8× (P=0.001), respectively.

Relative Rate of Absorption (RRA): This is a relationship between absorption and the time to achieve this absorption. This is calculated by: RRA=Cₘₐₓ/AUC₀₋₂₄. With these calculations the effect of Cₘₐₓ and AUC are compared. In these results (Table 2), 1.0 would be equivalent to CellSorb-Q10; >1.0=results inferior to CellSorb-Q10. RRAs for CellSorb-Q10, compared to R2 and R3 Ubiquinone showed a similar absorption rate. CellSorb-Q10 absorption had a rate equivalent to 1.8× that of the R1 Ubiquinone reference.

Relative Bioavailability (RB): Compares the effects of a single dose to be made for all products evaluated. RB=AUC_{0-24T}/AUC_{0-24R} (where T is CellSorb-Q10 and R would be the reference product). Both rate and degree of absorption play a critical role in the bioavailability. This relationship is taken into account in the relative bioavailability. In these results (Table 1), 1.0 would be equivalent to CellSorb-Q10; >1.0=results inferior to CellSorb-Q10. CellSorb-Q10 Relative Bioavailability (RB) was found to be bioequivalent to that of R1 and R2 Ubiquinone references. CellSorb-Q10 mean RB was significantly superior to that of R3 Ubiquinone (p=0.002). In addition, the mean responses for CellSorb-Q10 were 1.4× that of R2 Ubiquinone and 4.4× that of R3 Ubiquinone.

Repetitive Dosing: Many studies have shown that daily administration of CoQ₁₀ orally resulted in an additive, sequential increase in systemic CoQ₁₀ concentrations. In order to determine if treatments used in this study also had an additive effect, additional dosing (200 mg/day) was administered at time points 24 hrs & 48 hrs.

Table 3 shows the Repetitive Dosing responses for the reference products versus CellSorb-Q10. CellSorb-Q10 provided higher levels of CoQ10 (compared to the 3 reference products) that progressively increased with each day of Repetitive Dosing.

### Conclusions

This study was designed to evaluate the performance of CellSorb-Q10 with that of 3 reference products. Conclusive results can be summarized as follows:

In Pharmacokinetic Properties (mean Cₘₐₓ & AUC₀₋₂₄ₕᵣ) CellSorb-Q10 was found to be equivalent to results for R1 Ubiquinone and R2 Ubiquinone, but superior to the results for R3 Ubiquinone.

In Relative Degree of Absorption, CellSorb-Q10 was comparable in trending to 2 of the reference products and superior to that of R3 Ubiquinone by 2.8×.

In Relative Rate of Absorption, CellSorb-Q10 showed a rapid delivery of CoQ10 for metabolism within the body, which was superior to R1 Ubiquinone by 1.8×.

In Relative Bioavailability, CellSorb-Q10 was significantly superior in performance to R3 Ubiquinone by 4.4×.

In Repetitive Dosing, CellSorb-Q10 was noted to have higher average levels of daily CoQ10 by 1.2× of R1 Ubiquinone; by 1.3× of R2 Ubiquinone and 1.3× of R3 Ubiquinone.

The properties of Co Q10 delivery for CellSorb-Q10 were found to be very effective and superior by several criteria when compared to the 3 reference products.

TABLE 1
Relative Degree of Absorption and Bioavailability

| Pharmacokinetic | Relative Responses (Avg ± SE) for CellSorb-Q10 vs. | | |
|---|---|---|---|
| Relationships | R1 Ubiquinone | R2 Ubiquinone | R3 Ubiquinone |
| Relative Degree | 1.019 ± 0.219 | 1.203 ± 0.331 | 2.794 ± 0.598 * |
| of Absorption | | | |
| Relative | 0.812 ± 0.169 | 1.348 ± 0.395 | 4.423 ± 1.170 ¶ |
| Bioavailability | | | |

Significance:
* (p = 0.001);
¶ (p = 0.002)

TABLE 2
Relative Rates of Absorption

| | Absorption Rates (Avg ± SE) for | | | |
|---|---|---|---|---|
| Pharmacokinetic Relationship | CellSorb-Q10 | R1 Ubiquinone | R2 Ubiquinone | R3 Ubiquinone |
| Relative Rates of Absorption | 0.172 ± 0.034 | 0.094 ± 0.104 | 0.167 ± 0.033 | 0.170 ± 0.024 |

TABLE 3
Changes in CoQ10 Levels with Repetitive Dosing

| | | | | | Avg Daily | |
|---|---|---|---|---|---|---|
| | | | | | Value | % of |
| | Co Q10 Concentrations Avg ± SE (ng/ml) at | | | | (24-96 hrs) | CellSorb- |
| Treatment | 24 hrs | 48 hrs | 72 hrs | 96 hrs | ng/ml | Q10 |
| CellSorb- | 114.9 ± 25.0 | 309.2 ± 125.7 | 261.0 ± 81.8 | 100.1 ± 32.3 | 196.3 | 100.00 |
| Q10 | | | | | | |
| R1 | 143.3 ± 27.6 | 198.5 ± 39.0 | 206.0 ± 38.2 | 85.6 ± 25.0 | 158.4 | 80.7 |
| Ubiquinone | | | | | | |
| R2 | 88.3 ± 37.8 | 176.1 ± 41.8 | 227.4.0 ± 78.0 | 98.9 ± 45.6 | 147.7 | 75.2 |
| Ubiquinone | | | | | | |
| R3 | 19.1 ± 8.9 | 248.2 ± 148.4 | 233.6 ± 101.1 | 103.7 ± 42.1 | 151.2 | 77.0 |
| Ubiquinone | | | | | | |

TABLE 4

| | Mean ± SD (n = 10) | | | |
|---|---|---|---|---|
| | (Untransformed data) | | CV (%) | |
| PK | | Refer- | | Refer- |
| Parameter | Test | ence | Test | ence |
| (Units) | (T) | (R1) | (T) | (R1) |
| Cₘₐₓ | 326.96 ± 246.13 ^{NS} | 336.61 ± 144.23 | 75.28 | 42.85 |
| (ng/mL) | | | | |
| Tₘₐₓ | 6.00 ± 1.34 | 6.00 ± 1.94 | 75.87 | 27.77 |
| (h)* | | | | |
| AUC₀₋₂₄ | 3017.8 ± 2231.7 ^{NS} | 3971.8 ± 2047.1 | 73.95 | 51.54 |
| (ng · h/mL) | | | | |

^{NS} = not significantly different from reference

TABLE 5

| | Mean ± SD (n = 9) | | | |
|---|---|---|---|---|
| | (Untransformed data) | | CV (%) | |
| PK | | Refer- | | Refer- |
| Parameter | Test | ence | Test | ence |
| (Units) | (T) | (R2) | (T) | (R2) |
| Cₘₐₓ | 424.11 ± 437.10 ^{NS} | 366.28 ± 167.31 | 103.06 | 45.68 |
| (ng/mL) | | | | |
| Tₘₐₓ | 6.00 ± 1.00 | 6.00 ± 0.00 | 16.67 | 0.00 |
| (h) * | | | | |
| AUC₀₋₂₄ | 3436.7 ± 4261.5 ^{NS} | 3136.9 ± 2456.4 | 124.00 | 78.31 |
| (ng · h/mL) | | | | |

^{NS} = not significantly different from reference

TABLE 6

| | Mean ± SD (n = 9) | | | |
|---|---|---|---|---|
| | (Untransformed data) | | CV (%) | |
| PK | | Refer- | | Refer- |
| Parameter | Test | ence | Test | ence |
| (Units) | (T) | (R3) | (T) | (R3) |
| Cₘₐₓ | 341.76 ± 185.7 ¶ | 148.69 ± 65.28 | 54.33 | 43.90 |
| (ng/mL) | | | | |
| Tₘₐₓ | 6.22 ± 0.67 | 6.39 ± 2.52 | 10.77 | 39.4 |
| (h) * | | | | |
| AUC₀₋₂₄ | 3286.5 ± 1718.3 ¶ | 968.99 ± 427.37 | 52.28 | 44.10 |
| (ng · h/mL) | | | | |

* Median value was reported for Tₘₐₓ
¶ Significantly different from R3 Ubiquinone (p = 0.004)

### Example 2

### Relative Composition of CoQ10 in Commercially Available Products

There are numerous products currently on the market containing the compound CoQ10. These products are sold as being beneficial for promoting health in a variety of different ways (e.g heart health, antioxidant protection, increased cellular energy). However, CoQ10 is a lipophilic molecule and is not particularly well absorbed. Thus, one point of difference among products is their bioavailability. A number of commercial CoQ10 products promote themselves on this point, claiming that their product is better absorbed than a competitor's product.

CoQ10 exists in two forms—an oxidized form (the quinone) and a reduced form (the quinol). One company's website states that the reduced form is better absorbed than the oxidized form, and implies that their product is in this reduced form. Therefore, it was of interest to determine whether this product and several others contained the reduced form of CoQ10. It was also of interest to determine whether the amount of CoQ10 in the product was as claimed.

### Methods

CoQ10 content of the products was determined using high performance liquid chromatography (HPLC), with detection of the compound at 275 nm. Since a commercial source of the reduced form of CoQ10 is not readily available, the reduced form was prepared by reduction of the oxidized form with sodium borohydride. Quantitation was done by comparison with an authentic standard of CoQ10.

### Results

The table below shows for the three products tested the total amount of CoQ10 in a 100 mg softgel and the amount of each form (oxidized and reduced):

| | Amount | Amount | |
|---|---|---|---|
| | Reduced | Oxidized | Total CoQ10 amount (in |
| Product | Form (mg) | Form (mg) | mg) in 100 mg softgel |
| Qunol ™ Ultra | 0 | 125.6 | 125.6 |
| CoQ10 (R1) | | | |
| Q-sorb ™ | 0 | 121.4 | 121.4 |
| CoQ10 (R2) | | | |
| Nature Made ® | 0 | 118.8 | 118.8 |
| CoQ10 (R3) | | | |

As can be seen, none of the products contained reduced CoQ10. This is consistent with the label for the Qunol™ Ultra CoQ10 and Nature Made® CoQ10 products, which state they contain ubidecarenone, which is another name for the oxidized form of CoQ10.

All three products exceeded somewhat the amount of CoQ10 stated on the label. Presumably this is to insure they are well within the stated amount of the product. All products contained in excess the amount of CoQ10 stated on the label. The products contained only the oxidized form of CoQ10.

What is claimed is:

1. A composition comprising Coenzyme Q10 and a cetylated fatty acid.

2. The composition of claim 1, wherein the composition comprises a mixture of two or more cetylated fatty acids.

3. The composition of claim 1, wherein the cetylated fatty acid is selected from the group consisting of cetylated decanoic acid, cetylated lauric acid, cetylated myristic acid, cetylated myristoleic acid, cetylated palmitoleic acid, cetylated oleic acid, and cetylated stearic acid.

4. The composition of claim 1, further comprising a carrier or an excipient.

5. The composition of claim 5, wherein the carrier or excipient is selected from the group consisting of lecithin, olive oil, and tocophenol.

6. The composition of claim 1 comprising between 10-500 mg of Coenzyme Q10.

7. The composition of claim 1 comprising between 50-400 mg of Coenzyme Q10.

8. A pharmaceutical formulation comprising Coenzyme Q10 and a cetylated fatty acid.

9. The formulation of claim 8, wherein the formulation is in the form of a soft, sealed capsule.

10. The formulation of claim 9, wherein the capsule is made of gelatin and a plasticizer.

11. The formulation of claim 10, wherein the plasticizer is glycerol or sorbitol.

12. The formulation of claim 8 comprising between 10-500 mg of Coenzyme Q10.

13. The formulation of claim 8, wherein the formulation comprises a mixture of two or more cetylated fatty acids.

14. The formulation of claim 8, wherein the cetylated fatty acid is selected from the group consisting of cetylated decanoic acid, cetylated palmitic acid, cetylated lauric acid, cetylated myristic acid, cetylated myristoleic acid, cetylated palmitoleic acid, cetylated oleic acid, and cetylated stearic acid.

15. A method of increasing the systemic concentration of Coenzyme Q10 in an individual, comprising identifying an individual in need thereof and administering to the individual a composition comprising Coenzyme Q10 and a cetylated fatty acid.

16. The method of claim 15, wherein the Coenzyme Q10 is administered at a dose of between 10-500 mg.

17. The method of claim 15, wherein the composition is administered once a day.

18. The method of claim 15, wherein the composition comprises a mixture of two or more cetylated fatty acids.

19. The method of claim 15, wherein the cetylated fatty acid is selected from the group consisting of cetylated decanoic acid, cetylated palmitic acid, cetylated lauric acid, cetylated myristic acid, cetylated myristoleic acid, cetylated palmitoleic acid, cetylated oleic acid, and cetylated stearic acid.

20. The method of claim 15, wherein the composition is administered in a soft, sealed capsule.
